(19) United States
(12) Patent Application Publication
(10) Pub. No.: US 2018/0296065 A1
(43) Pub. Date: Oct. 18, 2018

(54) ENDOSCOPE DEVICE

(71) Applicant: OLYMPUS CORPORATION, Tokyo (JP)

(72) Inventors: Mitsunobu ONO, Tokyo (JP); Kenji NUMATA, Tokyo (JP)

(73) Assignee: OLYMPUS CORPORATION, Tokyo (JP)

(21) Appl. No.: 16/018,248

(22) Filed: Jun. 26, 2018

Related U.S. Application Data
(63) Continuation of application No. PCT/JP2017/000239, filed on Jan. 6, 2017.

(30) Foreign Application Priority Data
Jan. 12, 2016 (JP) 2016-003611

(51) Int. Cl. A61B 1/00

(52) U.S. Cl. CPC A61B 1/00009; A61B 1/00006; A61B 1/051; A61B 1/00013; A61B 1/00018

(57) ABSTRACT

An endoscope device includes a tip section including an imaging element for outputting a pixel signal of a captured image and configured to be inserted into an object, and a flexible section including a first serial signal transmission path along which a setting related to photographing is transmitted to the imaging element via first serial communication and a second serial signal transmission path along which the pixel signal is transmitted via second serial communication and configured to guide the tip section into the object, wherein the second serial signal transmission path includes an equalizer circuit configured to correct frequency characteristics of a serial signal and a limiting amplifier circuit configured to amplify the serial signal corrected by the equalizer circuit, and wherein the equalizer circuit and the limiting amplifier circuit are connected on the same substrate surface.

This application is a continuation application based on a PCT International Patent Application No. PCT/JP2017/000239, filed Jan. 6, 2017, whose priority is claimed on Japanese Patent Application No. 2016-003611, filed Jan. 12, 2016, the content of the PCT International Patent Application and the Japanese Patent Application are incorporated herein by reference.

### TECHNICAL FIELD

The present invention relates to an endoscope device used by inserting an insertion section into a specimen.

### BACKGROUND ART

Conventionally, endoscope devices configured to insert an elongated insertion section into a specimen and photograph the inside of the specimen with an imaging element provided in a tip section located at a tip of the insertion section have been practically used in industrial fields and medical fields. In the conventional endoscope devices, a charge coupled device (CCD) image sensor is mounted as an imaging element, and a signal of an image within a specimen imaged by the CCD image sensor is transmitted to a main body section through a signal cable provided in the insertion section.

For example, in Japanese Unexamined Patent Application, First Publication No. 2012-115531, an endoscope device having a configuration in which an analog image signal output from a CCD image sensor is transmitted to a main body section by a serial cable via correlated double sampling (CDS) and a buffer is disclosed. In the endoscope device disclosed in Japanese Unexamined Patent Application, First Publication No. 2012-115531, an analog image signal output from the CCD image sensor is transmitted as an analog signal to the main body section as it is.

Also, for example, Japanese Unexamined Patent Application, First Publication No. 2011-036585 discloses an endoscope device having a configuration in which an analog image signal output from a CCD image sensor is converted into parallel digital signals through analog/digital conversion (A/D conversion), the parallel digital signals are further converted into a serial digital signal by a serializer, and the serial digital signal is transmitted to a main body section. In the endoscope device disclosed in Japanese Unexamined Patent Application, First Publication No. 2011-036585, the transmitted serial digital signal is returned to the original parallel digital signals by a deserializer provided in the main body section. That is, in the endoscope device disclosed in Japanese Unexamined Patent Application, First Publication No. 2011-036585, the analog image signal output from the CCD image sensor is returned to the parallel digital signals obtained through analog/digital conversion.

Also, an endoscope device using a complementary metal-oxide semiconductor (CMOS) image sensor in place of the CCD image sensor has recently been put to practical use. In the endoscope device having the CMOS image sensor mounted thereon, it is possible to transmit a signal of an image inside the imaged specimen to the main body section in a digital signal.

For example, as in an endoscope device disclosed in Japanese Unexamined Patent Application, First Publication No. 2010-051503, an endoscope device configured to transmit a control signal for a component included in a CMOS image sensor as a digital serial signal is disclosed. In the endoscope device disclosed in Japanese Unexamined Patent Application, First Publication No. 2010-051503, a command for changing a driving current of a light-emitting element for converting an electric signal of an image into an optical signal is transmitted to a receiver provided in the CMOS image sensor via an inter-integrated circuit (I2C) cable. Here, because it is possible to transmit various commands and control signals through two signal cables, signal transmission based on I2C is an effective transmission method for narrowing a diameter of an insertion section.

However, in a general endoscope device, a total length of an insertion section extends to several meters. An I2C transmission scheme is a standard in consideration of short-distance signal transmission. Thus, the I2C transmission scheme is not suitable for application to an insertion section of an endoscope device used in an environment where a signal transmission distance is long and an amount of noise is large. Thus, when signal transmission based on I2C is performed in an endoscope device configured to transmit signals (digital signals) through a signal cable provided within the insertion section, many countermeasures against noise are required, for example, to satisfy a requirement of electro-magnetic compatibility (EMC). In other words, in an endoscope device in which a total length of the insertion section extends to several meters (in particular, an endoscope device having an insertion section with a length of several tens of meters for use in industrial fields), it is necessary to take many countermeasures against noise such as EMC to apply the I2C transmission scheme.

Also, in an industrial endoscope device, for example, it is conceivable to strengthen a shield by increasing the diameter of the I2C cable to satisfy EMC by applying the I2C transmission scheme. However, in an industrial endoscope device, if the I2C cable is thickened, the diameter of the insertion section also becomes thick, and it is not easy to handle the insertion section. Thus, a method of performing a countermeasure against noise such as EMC by thickening the I2C cable in an industrial endoscope device is not a practical method.

### SUMMARY OF INVENTION

According to a first aspect of the present invention, an endoscope device includes a tip section including an imaging element for outputting a pixel signal according to a captured image of a subject and configured to be inserted into an object; and a flexible section including a first serial signal transmission path along which a setting related to photographing is transmitted to the imaging element via first serial communication and a second serial signal transmission path along which the pixel signal output by the imaging element is transmitted via second serial communication and configured to guide the tip section into the object, wherein the second serial signal transmission path includes an equalizer circuit configured to correct frequency characteristics of a serial signal for transmitting the pixel signal via the second serial communication and a limiting amplifier circuit configured to amplify the serial signal corrected by the equalizer circuit, and wherein the equalizer circuit and the limiting amplifier circuit are connected on the same substrate surface.

According to a second aspect of the present invention, the endoscope device of the above-described first aspect may include a main body section including an image processing section configured to perform image processing on the pixel signal transmitted according to the second serial communication.

According to a third aspect of the present invention, the endoscope device of the above-described second aspect may further include a connector section configured to electrically connect the first serial signal transmission path and the second serial signal transmission path provided in the flexible section to corresponding components provided in the main body section.

According to a fourth aspect of the present invention, in the endoscope device of the above-described third aspect, the equalizer circuit and the limiting amplifier circuit may be arranged in the connector section.

According to a fifth aspect of the present invention, in the endoscope device of the above-described first aspect, the equalizer circuit may correct a signal level of the input serial signal so that an attenuation rate of a signal level of a corrected signal becomes smaller when a frequency band of the input signal is higher, and that the attenuation rate of the signal level of the corrected signal becomes greater when the frequency band of the input signal is lower.

According to a sixth aspect of the present invention, in the endoscope device of the above-described first aspect, a cable for transmitting the pixel signal and the equalizer circuit in the second serial signal transmission path may be connected on the same substrate surface.

According to a seventh aspect of the present invention, in the endoscope device of the above-described third aspect, a cable for transmitting the pixel signal and the equalizer circuit may be arranged in the connector section.

According to an eighth aspect of the present invention, an endoscope device includes a tip section including an imaging element for outputting a pixel signal according to a captured image of a subject and configured to be inserted into an object; and a flexible section including a first serial signal transmission path along which a setting related to photographing is transmitted to the imaging element via first serial communication and a second serial signal transmission path along which the pixel signal output by the imaging element is transmitted via second serial communication and configured to guide the tip section into the object, wherein the second serial signal transmission path includes an equalizer circuit configured to correct frequency characteristics of a serial signal for transmitting the pixel signal via the second serial communication, and wherein a cable for transmitting the pixel signal and the equalizer circuit are connected on the same substrate surface.

According to a ninth aspect of the present invention, the endoscope device of the above-described eighth aspect may include a main body section including an image processing section configured to perform image processing on the pixel signal transmitted according to the second serial communication.

According to a tenth aspect of the present invention, in the endoscope device of the above-described eighth aspect, the second serial signal transmission path may include a limiting amplifier circuit configured to amplify the serial signal corrected by the equalizer circuit.

According to an eleventh aspect of the present invention, in the endoscope device of the above-described tenth aspect, the equalizer circuit may correct a signal level of the input serial signal so that an attenuation rate of a signal level of a corrected signal becomes smaller when a frequency band of the input signal is higher, and that the attenuation rate of the signal level of the corrected signal becomes greater when the frequency band of the input signal is lower.

According to a twelfth aspect of the present invention, in the endoscope device of the above-described tenth aspect, the equalizer circuit and the limiting amplifier circuit in the second serial signal transmission path may be connected on the same substrate surface.

According to a thirteenth aspect of the present invention, the endoscope device of the above-described ninth aspect may further include a connector section configured to electrically connect the first serial signal transmission path and the second serial signal transmission path provided in the flexible section to corresponding components provided in the main body section.

According to a fourteenth aspect of the present invention, in the endoscope device of the above-described thirteenth aspect, the equalizer circuit and a limiting amplifier circuit configured to amplify the serial signal corrected by the equalizer circuit may be arranged in the connector section.

According to a fifteenth aspect of the present invention, in the endoscope device of the above-described thirteenth aspect, the cable and the equalizer circuit may be arranged in the connector section.

### BRIEF DESCRIPTION OF DRAWINGS

FIG. 1 is a block diagram showing an example of a schematic configuration of an endoscope device according to a first embodiment of the present invention.

FIG. 2 is a circuit diagram showing an example of an equalizer circuit provided in the endoscope device of the first embodiment of the present invention.

FIG. 3 is a diagram showing an example of frequency characteristics of the equalizer circuit provided in the endoscope device of the first embodiment of the present invention.

FIG. 4 is a block diagram showing an example of a schematic configuration of an endoscope device according to a second embodiment of the present invention.

FIG. 5 is a block diagram showing an example of a schematic configuration of an endoscope device according to a third embodiment of the present invention.

### DESCRIPTION OF EMBODIMENTS

### First Embodiment

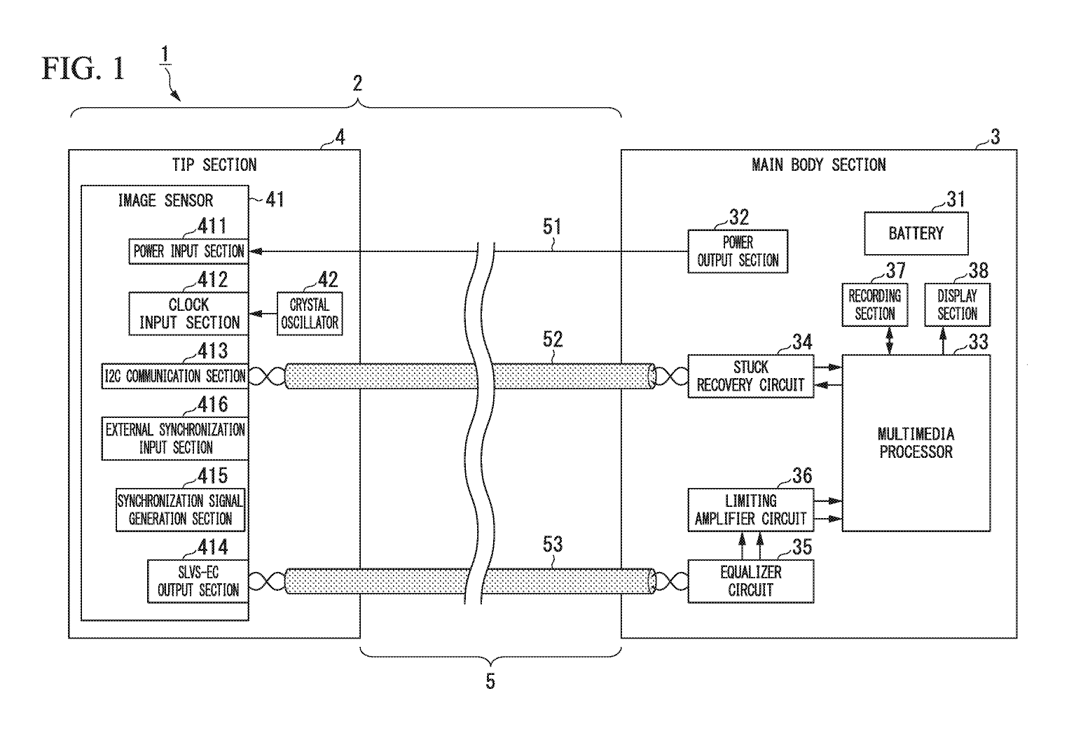

Hereinafter, embodiments of the present invention will be described with reference to the drawings. In the following description, a case in which an endoscope device of the present invention is an industrial endoscope device will be described. FIG. 1 is a block diagram showing an example of a schematic configuration of an endoscope device according to a first embodiment of the present invention. In FIG. 1, an endoscope device 1 includes an elongated insertion section 2 and a main body section 3. The insertion section 2 is configured to include a tip section 4 having an imaging element and a flexible section 5 which is a cord for guiding the tip section 4 into a specimen.

In the endoscope device 1, a pixel signal obtained through photographing performed by an imaging element provided in the tip section 4 is transmitted to the main body section 3 via the flexible section 5. Also, in the endoscope device 1, a movement and a direction of the tip section 4 when the tip section 4 is guided by the flexible section 5 and inserted into the specimen, and a subject photographing operation of the imaging element provided within the tip section 4 are operated from the main body section 3 via the flexible section 5. In the endoscope device 1, a video (an image) generated by processing the pixel signal transmitted from the tip section 4, by the main body section 3 is displayed. Also, in the endoscope device 1, the video (the image) generated through processing by the main body section 3 is recorded. When the endoscope device 1 does not perform photographing within the specimen, for example, the insertion section 2 is wound around a drum section (not shown) attached to the main body section 3 and stored in the endoscope device 1.

The tip section 4 includes an image sensor 41 serving as an imaging element and a crystal oscillator 42. The flexible section 5 includes a power signal line 51, an I2C serial signal transmission path 52, and an SLVS-EC serial signal transmission path 53. The main body section 3 includes a battery 31, a power output section 32, a multimedia processor 33, a stuck recovery circuit 34, an equalizer circuit 35, a limiting amplifier circuit 36, a recording section 37, and a display section 38. Also, the multimedia processor 33 may also be referred to as a system on chip (SoC).

Here, each component included in the endoscope device 1 will be described in detail. First, each component provided in the tip section 4 will be described in detail.

The crystal oscillator 42 oscillates an operation clock signal of a predetermined frequency required when the image sensor 41 operates and supplies the oscillated operation clock signal to the image sensor 41.

Also, the crystal oscillator 42 does not need to oscillate an operation clock signal synchronized with a clock signal when the main body section 3 operates or to supply the oscillated operation clock signal to the image sensor 41. That is, in the endoscope device 1, for example, the crystal oscillator 42 does not need to oscillate the operation clock signal synchronized with the synchronization signal or the like output from the main body section 3. Thus, the endoscope device 1 is configured so that a high-frequency operation clock signal is not transmitted from the main body section 3 to the tip section 4. Accordingly, in the endoscope device 1, it is unnecessary to provide a thick coaxial transmission line in the flexible section 5 to prevent a waveform shaping circuit and a waveform of the operation clock signal provided in the conventional endoscope device from being degraded and it is possible to reduce the size of the tip section 4.

The image sensor 41 is a CMOS image sensor configured to operate on the basis of a clock signal oscillated by the crystal oscillator 42. The image sensor 41 includes a pixel array section (not shown) for outputting pixel signals corresponding to an image within the subject in the imaged specimen, a power input section 411, a clock input section 412, an inter-integrated circuit (I2C) communication section 413, a scalable low voltage signaling with embedded clock (SLVS-EC) output section 414, a synchronization signal generation section 415, and an external synchronization input section 416.

The power input section 411 converts power supplied from the main body section 3 via the power signal line 51 provided in the flexible section 5 into a voltage required by each component in the image sensor 41. The power input section 411 supplies the power of each voltage obtained through the conversion to each component.

The clock input section 412 converts the operation clock signal input from the crystal oscillator 42 into a frequency required by each component in the image sensor 41. The clock input section 412 supplies the converted clock signal to each component.

The I2C communication section 413 performs serial communication (hereinafter referred to as “I2C serial communication”) through the I2C bus with the main body section 3 via the I2C serial signal transmission path 52 provided in the flexible section 5. The I2C serial communication is performed according to a transmission path (the I2C serial signal transmission path 52) including two signal lines. The I2C communication section 413 outputs function activation and operation settings of the image sensor 41 input from the main body section 3 to the corresponding components according to the

I2C serial communication. For example, various settings related to photographing such as an electronic shutter, an exposure time, and a photographing interval (a so-called frame rate) when the pixel array section (not shown) photographs the subject (hereinafter referred to as “photographing mode settings”) are transmitted from the main body section 3 to the I2C communication section 413 according to the I2C serial communication. When the photographing mode settings transmitted from the main body section 3 via the I2C serial signal transmission path 52 are received, the I2C communication section 413 outputs information of the received photographing mode settings to the pixel array section (not shown). Thereby, the pixel array section (not shown) performs photographing according to the information of the photographing mode settings output from the I2C communication section 413, and outputs pixel signals obtained through photographing. Because a communication method of the I2C serial communication in the I2C communication section 413 is similar to that of the serial communication using the existing I2C bus, a detailed description thereof will be omitted.

The SLVS-EC output section 414 converts a pixel signal (for example, a RAW signal) output through photographing performed by the pixel array section (not shown) into a serial signal of a type of serial communication using SLVS-EC (hereinafter referred to as “SLVS-EC serial communication”). The SLVS-EC output section 414 transmits the serial signal obtained through the conversion to the main body section 3 via the SLVS-EC serial signal transmission path 53. That is, the image sensor 41 is a CMOS image sensor configured to output pixel signals corresponding to an image of the imaged subject within the specimen according to the SLVS-EC serial communication. The SLVS-EC serial communication is also performed through a transmission path (the SLVS-EC serial signal transmission path 53) including two signal lines.

Also, the pixel signals transmitted by the SLVS-EC output section 414 to the main body section 3 according to the SLVS-EC serial communication are digital signals. Thus, the pixel signals output through photographing performed by the pixel array section (not shown) are converted into parallel digital signals by an analog/digital conversion (A/D conversion) circuit (not shown) and then input to the SLVS-EC output section 414. Accordingly, the SLVS-EC output section 414 converts the input pixel signals, which are parallel digital signals, into a serial digital signal of the type of SLVS-EC serial communication, and transmits the serial digital signal to the main body section 3. However, in the following description, for ease of description, the SLVS-EC output section 414 converts pixel signals output through photographing performed by the pixel array section (not shown) into a serial signal of the type of SLVS-EC serial communication and transmits the serial signal to the main body section 3.

The synchronization signal generation section 415 generates a synchronization signal (a horizontal synchronization signal or a vertical synchronization signal) representing a timing at which the pixel signals obtained through photographing performed by the pixel array section (not shown) are output on the basis of the clock signal supplied from the clock input section 412. The synchronization signal generation section 415 outputs the generated synchronization signals to the pixel array section (not shown). Thereby, the pixel array section (not shown) outputs the pixel signals obtained through photographing to the SLVS-EC output section 414 at timing synchronized with each input synchronization signal.

In the endoscope device 1, each synchronization signal output from the synchronization signal generation section 415 is superimposed on the serial signal of the SLVS-EC serial communication and is transmitted to the main body section 3. Thus, the synchronization signal generation section 415 also outputs each generated synchronization signal to the SLVS-EC output section 414. When the pixel signals output from the pixel array section (not shown) are converted into a serial signal in the type of SLVS-EC serial communication, the SLVS-EC output section 414 superimposes each synchronization signal output from the synchronization signal generation section 415 thereon. That is, the SLVS-EC output section 414 transmits the serial signal of the type of SLVS-EC serial communication in which each synchronization signal output from the synchronization signal generation section 415 is embedded as the clock signal to be embedded in the SLVS-EC serial communication to the main body section 3 via the SLVS-EC serial signal transmission path 53. Thereby, in the endoscope device 1, the synchronization signal (the horizontal synchronization signal or the vertical synchronization signal) is transmitted to the main body section 3 together with the pixel signals according to the SLVS-EC serial communication. In the endoscope device 1, the main body section 3 performs various processes on the pixel signals in synchronization with the synchronization signal transmitted together with the pixel signals. That is, in the endoscope device 1, the image sensor 41 performs various processes on the pixel signals in synchronization with the timing of the horizontal synchronization signal or the vertical synchronization signal at which the pixel signals are output. Because the communication method of the SLVS-EC serial communication in the SLVS-EC output section 414 is similar to that of the serial communication using the existing SLVS-EC, a detailed description thereof will be omitted.

The external synchronization input section 416 is an input section to which the synchronization signal (the horizontal synchronization signal or the vertical synchronization signal) from the outside is input. When each synchronization signal is input from the outside to the external synchronization input section 416, each synchronization signal input from the outside (hereinafter referred to as an “external synchronization signal”) is output to the pixel array section (not shown). Thereby, the pixel array section (not shown) outputs the pixel signals obtained through photographing to the SLVS-EC output section 414 at the timing synchronized with each input external synchronization signal. That is, when the external synchronization signal is input to the external synchronization input section 416, the image sensor 41 is a CMOS image sensor that operates in synchronization with the input external synchronization signal. The external synchronization signal is transmitted from the main body section 3 via, for example, an external synchronization signal line (not shown) provided in the flexible section 5.

Also, even when the image sensor 41 operates in synchronization with the external synchronization signal, the external synchronization input section 416 also outputs the input external synchronization signal to the SLVS-EC output section 414. Thereby, when the pixel signals output from the pixel array section (not shown) are converted into a serial signal of the type of SLVS-EC serial communication, the SLVS-EC output section 414 superimposes each external synchronization signal output from the external synchronization input section 416 thereon and transmits the superimposed signal to the main body section 3.

In the configuration of the endoscope device 1 shown in FIG. 1, a configuration in which the image sensor 41 is a CMOS image sensor operating in synchronization with the synchronization signal generated by the synchronization signal generation section 415 is shown. Accordingly, the endoscope device 1 is configured so that the main body section 3 operates in synchronization with the synchronization signal generated by the synchronization signal generation section 415. However, as described above, the image sensor 41 can operate in synchronization with the external synchronization signal. However, in this case, as described above, it is necessary to provide an external synchronization signal line (not shown) for separately transmitting the external synchronization signal to the image sensor 41 in the flexible section 5. Thus, in the endoscope device 1 configured to operate in synchronization with the external synchronization signal, it is conceivable that the number of signal lines provided in the flexible section 5 increases and the exterior of the flexible section 5 becomes thick. Accordingly, as shown in FIG. 1, the image sensor 41 is preferably configured to operate in synchronization with the synchronization signal generated by the synchronization signal generation section 415. Then, in the image sensor 41, the SLVS-EC output section 414 transmits the synchronization signals together with the pixel signals. Thereby, in the endoscope device 1, even though a signal line corresponding to each synchronization signal is not provided in the flexible section 5 to separately transmit each synchronization signal generated by the synchronization signal generation section 415 provided in the image sensor 41 to the main body section 3, photographing in the image sensor 41 and processing in the main body section 3 can be operated in synchronization with each other.

Next, each component provided in the main body section 3 will be described in detail.

The battery 31 supplies power for driving the components provided in the main body section 3 and the components provided in the tip section 4. For example, the battery 31 is a rechargeable battery such as a lithium ion secondary battery.

The power output section 32 supplies power output from the battery 31 to each component provided in the tip section 4 via the power signal line 51 provided in the flexible section 5. FIG. 1 shows a state in which the power output section 32 supplies power to the power input section 411 provided in the image sensor 41 within the tip section 4 via the power signal line 51.

The multimedia processor 33 is a control section configured to perform overall control in the endoscope device 1. For example, the multimedia processor 33 transmits various settings related to function activation or a photographing operation of the image sensor 41 provided in the tip section 4 indicated by a user of the endoscope device 1 operating a dedicated operation device such as an operation section (not shown) or a remote control terminal to the I2C communication section 413 provided in the image sensor 41 within the tip section 4 according to the I2C serial communication and controls photographing of a subject within the specimen in the endoscope device 1. That is, the multimedia processor 33 transmits the photographing mode setting in the endoscope device 1 to the I2C communication section 413 provided in the image sensor 41 within the tip section 4 according to the I2C serial communication, and controls the photographing of the subject within the specimen in the endoscope device 1.

Also, the multimedia processor 33 is an image processing section configured to perform various types of predetermined image processing on the pixel signals (for example, RAW signals) obtained through photographing performed by the pixel array section (not shown) provided in the image sensor 41 within the tip section 4 and transmitted according to SLVS-EC serial communication and to generate an image of the subject within the imaged specimen. For example, the multimedia processor 33 generates an image (a still image or a moving image) for recording by performing image processing for recording on the pixel signals transmitted from the image sensor 41 and causes the generated image for recording to be recorded on the recording section 37. Also, for example, the multimedia processor 33 performs image processing for display on the pixel signals transmitted from the image sensor 41 to generate an image (a still image or a moving image) for display and cause the generated image for display to be output to the display section 38 for display. The multimedia processor 33 also performs image processing of reading an image (a still image or a moving image) for recording recorded on the recording section 37 and outputting the image to the display section 38 for display.

The recording section 37 records data of the image of the subject within the specimen imaged by the endoscope device 1. Also, although the recording section 37 is shown as a component embedded in the main body section 3 in FIG. 1, the recording section 37 may be, for example, a recording medium of a configuration capable of being attached to or detached from the main body section 3 such as an SD memory card or Compact Flash (registered trademark) (CF).

The display section 38 displays an image of the subject in the specimen imaged by the endoscope device 1. For example, the display section 38 includes a display device such as a liquid crystal display (LCD). Although the display section 38 is shown as a component mounted on the main body section 3 in FIG. 1, the display section 38 may be an external display device connected to the main body section 3, i.e., a display device having a configuration capable of being attached to or detached from the main body section 3.

The stuck recovery circuit 34 is connected to an end of the main body section 3 side of the I2C serial signal transmission path 52, and relays I2C serial communication between the I2C communication section 413 provided in the image sensor 41 within the tip section 4 and the multimedia processor 33. Also, the stuck recovery circuit 34 is a stuck bus recovery circuit for monitoring the state of the I2C serial communication and enabling the I2C serial communication to be performed normally. The stuck recovery circuit 34 is a component provided in consideration of a case in which much external noise enters the I2C serial signal transmission path 52 provided in the flexible section 5 of the elongated insertion section 2 and the I2C serial communication is stopped (stuck), for example, when the endoscope device 1 is used in a poor environment such as a factory having much electromagnetic noise. The stuck recovery circuit 34 determines whether or not the I2C serial communication is stopped by monitoring the state of the I2C serial communication, and performs a process of causing the stopped I2C serial communication to be recovered (resumed) if it is determined that the I2C serial communication is stopped.

More specifically, for example, a case in which a time in which a communication clock signal is stopped in the I2C serial communication due to external noise or the like is greater than or equal to a predetermined time is conceivable. In this case, the stuck recovery circuit 34 determines that the I2C serial communication is stopped. When it is determined that the I2C serial communication is stopped, the stuck recovery circuit 34 temporarily blocks the transmission path of the I2C serial communication, i.e., the I2C serial signal transmission path 52. The stuck recovery circuit 34 adds a predetermined number of automatically generated communication clock signals to the I2C serial signal transmission path 52 and therefore causes the I2C serial communication to be recovered (resumed) between the I2C communication section 413 provided in the image sensor 41 that is stopped and the multimedia processor 33. That is, the stuck recovery circuit 34 causes the I2C serial communication to be recovered (resumed) by operating a communication clock signal in the serial signal of the type of I2C serial communication (a so-called clock signal SCL generally serving as a reference in the I2C serial communication).

Thereby, the diameter of the insertion section 2 can be narrowed so that the operation is performed using two signal lines in the endoscope device 1 and it is possible to improve noise immunity of the I2C serial communication having characteristics vulnerable to external noise in a state in which the diameter of the insertion section 2 is narrowed by providing the stuck recovery circuit 34. That is, because it is necessary to lower the impedance of an electric wire by doubly shielding each signal line of the transmission path of the I2C serial communication or thickening the signal line itself to improve the noise immunity of the I2C serial communication in the conventional endoscope device, it is not easy to narrow the diameter of the transmission path of the I2C serial communication. On the other hand, both a process of reducing a countermeasure such as double shielding or thickening of a signal line required in the conventional endoscope device and narrowing the diameter of the I2C serial signal transmission path 52 and a process of improving noise immunity of the I2C serial communication are easily implemented by providing the stuck recovery circuit 34 in the endoscope device 1.

Also, the stuck recovery circuit 34 includes a function of an amplification circuit for amplifying a signal component of each serial signal (hereinafter referred to as an “I2C serial signal”) of the I2C serial communication attenuated in the I2C serial signal transmission path 52, for example, as in the I2C driver circuit. Also, because the configuration and function of the stuck recovery circuit 34 are similar to those of the existing stuck bus recovery circuit, a detailed description thereof will be omitted.

Also, although the stuck recovery circuit 34 is shown as a component mounted on the main body section 3 and connected to the multimedia processor 33 in FIG. 1, the stuck recovery circuit 34 may perform a function installed in the multimedia processor 33. That is, the stuck recovery circuit 34 may be a component included in the multimedia processor 33. Also, the stuck recovery circuit 34 may be configured to switch whether or not to enable the function of amplifying the signal component of the I2C serial signal in accordance with the length of the I2C serial signal transmission path 52. That is, the stuck recovery circuit 34 may be configured to enable a function of amplifying the signal component of each I2C serial signal only when the length of the I2C serial signal transmission path 52 is long. In other words, the stuck recovery circuit 34 may have a configuration in which the function of amplifying the signal component of each I2C serial signal is not provided, and the endoscope device 1 may have a configuration in which the stuck recovery circuit 34 of the configuration and the I2C driver circuit are provided in the main body section 3 when the length of the I2C serial signal transmission path 52 is long.

The equalizer circuit 35 is a circuit connected to an end of the main body section 3 side of the SLVS-EC serial signal transmission path 53 and configured to correct frequency characteristics of a serial signal of SLVS-EC serial communication transmitted from the SLVS-EC output section 414 provided in the image sensor 41 within the tip section 4. The equalizer circuit 35 outputs the serial signal of the SLVS-EC serial communication after correcting the frequency characteristics to the limiting amplifier circuit 36.

Because each pixel signal obtained through photographing performed by the image sensor 41 is transmitted via the SLVS-EC serial signal transmission path 53 provided in the flexible section 5 of the elongated insertion section 2 in the endoscope device 1 as described above, the attenuation of the serial signal of the SLVS-EC serial communication (hereinafter referred to as an “SLVS-EC serial signal”) increases as the number of high-frequency signal components increases. Thus, in the endoscope device 1, a state in which the waveform of the SLVS-EC serial signal is distorted (generally, a state opposite an “eye-pattern open state” in which quality of the waveform is high in two-line serial communication) is reached. The equalizer circuit 35 is a component provided to correct an amount of attenuation of the signal component which differs according to the frequency when transmission is performed via the SLVS-EC serial signal transmission path 53 so that the waveform of the SLVS-EC serial signal has a similar signal level in all frequency bands. That is, in general, the equalizer circuit 35 is a component provided to be in an “eye-pattern open state” in which quality of the waveform is high. In the endoscope device 1, it is possible to more accurately transmit a pixel signal of a high-frequency component required by the multimedia processor 33 for performing image processing according to the SLVS-EC serial communication by setting a high-quality state by improving the waveform of the SLVS-EC serial signal transmitted by the equalizer circuit 35 via the SLVS-EC serial signal transmission path 53.

Also, when the equalizer circuit 35 and each wire rod (cable) constituting the SLVS-EC serial signal transmission path 53 are connected in a substrate on which each component constituting the main body section 3 is mounted (hereinafter referred to as a “main body substrate”) in the endoscope device 1, a substrate surface on which the equalizer circuit 35 is mounted (soldered) and a substrate surface on which each cable constituting the SLVS-EC serial signal transmission path 53 is soldered are the same substrate surface. That is, in the endoscope device 1, the equalizer circuit 35 and each cable constituting the SLVS-EC serial signal transmission path 53 are connected on the same substrate surface of the main body substrate. Thereby, in the endoscope device 1, it is possible to ensure the quality of the waveform of the SLVS-EC serial signal without changing the characteristic impedance of the signal line between the equalizer circuit 35 and the SLVS-EC serial signal transmission path 53.

The equalizer circuit 35 performs correction so that a similar signal level is formed in all frequency bands to improve a waveform of an SLVS-EC serial signal by relatively outputting a signal component of a high-frequency band with a larger amount of attenuation as it is and attenuating a signal component of a low-frequency component with a small amount of attenuation to output the attenuated signal component when transmission is performed via the SLVS-EC serial signal transmission path 53. The equalizer circuit 35 includes, for example, an RLC circuit in which a resistor (R), a coil (L), and a capacitor (C) are combined. That is, the equalizer circuit 35 includes a filter circuit.

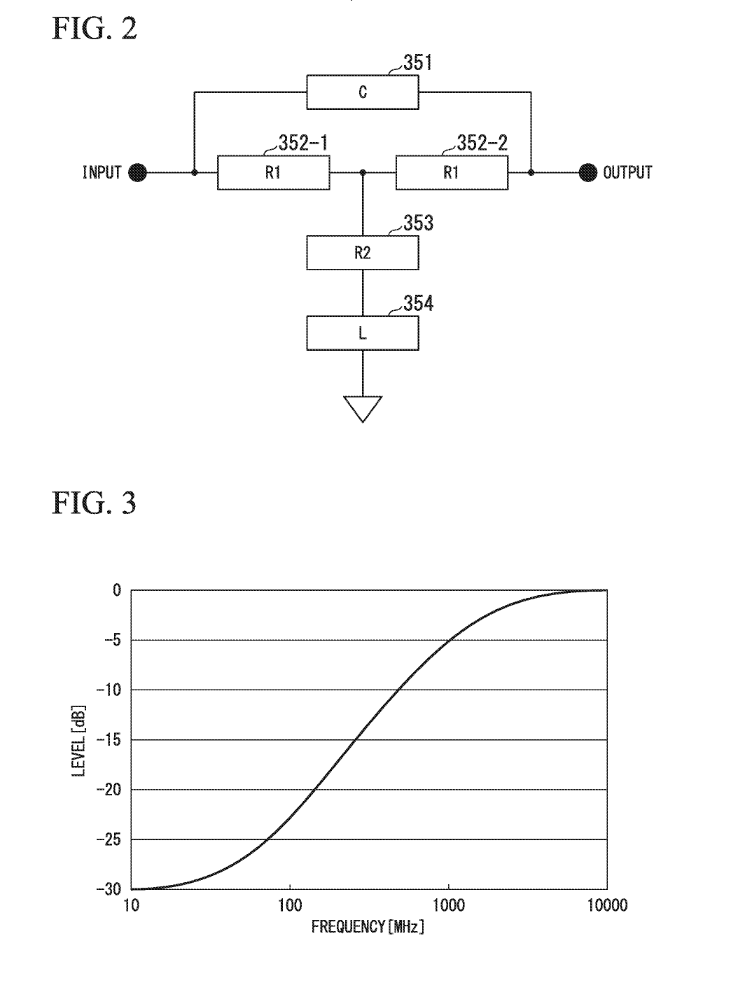

Here, an example of the circuit configuration of the equalizer circuit 35 and an example of the frequency characteristic of the equalizer circuit 35 will be described in detail. FIG. 2 is a circuit diagram showing an example of the equalizer circuit 35 provided in the endoscope device 1 according to the first embodiment of the present invention. Also, FIG. 3 is a diagram showing an example of the frequency characteristic of the equalizer circuit 35 provided in the endoscope device 1 of the first embodiment of the present invention.

In FIG. 2, the equalizer circuit 35 includes a capacitor (C) 351, two first resistors (R1) 352-1 and a first resistor (R1) 352-2, a second resistor (R2) 353, and a coil (L) 354. More specifically, a first terminal of the capacitor 351 and a first terminal of the first resistor 352-1 are connected to each other and serve as an input terminal of the equalizer circuit 35. A second terminal of the first resistor 352-1 is connected to a first terminal of the first resistor 352-2 and a first terminal of the second resistor 353. A second terminal of the second resistor 353 is connected to a first terminal of the coil 354. A second terminal of the coil 354 is grounded. A second terminal of the capacitor 351 and a second terminal of the first resistor 352-2 are connected to each other and serve as an output terminal of the equalizer circuit 35.

According to such a configuration, the equalizer circuit 35 shown in FIG. 2 is configured as a filter circuit having an S-shaped frequency characteristic (a filter characteristic) which is a combination of a low pass filter (LPF) and a high pass filter (HPF) as shown in FIG. 3. More specifically, as shown in FIG. 3, the frequency characteristic of the equalizer circuit 35 is a characteristic that the signal level of the output signal becomes similar to the signal level of the input signal when the frequency band is high and the signal level of the input signal becomes an attenuated level when the frequency band is low. In other words, the equalizer circuit 35 has a frequency characteristic that an attenuation rate of the signal level of the signal to be output is decreased when the frequency band is high and the attenuation rate of the signal level of the signal to be output is increased when the frequency band is low.

In the endoscope device 1, the equalizer circuit 35 having such a frequency band improves the waveform of the SLVS-EC serial signal transmitted (transmitted) via the SLVS-EC serial signal transmission path 53 and extracts a pixel signal of the high-frequency component required when the multimedia processor 33 performs image processing. That is, in the endoscope device 1, the equalizer circuit 35 extracts pixel signals with similar signal levels in all frequency bands by relatively outputting a signal component of an SLVS-EC serial signal of a high-frequency band with a larger amount of attenuation as it is and attenuating a signal component of a low-frequency component with a small amount of attenuation to output the attenuated signal component as described above.

Also, as a method of extracting pixel signals with similar signal levels in all the frequency bands, a plurality of methods other than the method performed by the equalizer circuit 35 are conceivable. For example, there is a method using technology called pre-emphasis or de-emphasis. These methods are technology in which signals are transmitted by increasing (emphasizing) the signal level of the frequency band attenuated according to a transmission path in advance or attenuating the signal level of the frequency band that is not attenuated according to a transmission path in advance at a transmission side from which serial signals are output and therefore serial signals having similar signal levels can be received in all frequency bands at a reception side to which the serial signals are input. However, when the technology of pre-emphasis or de-emphasis is applied to an endoscope device, more parts may be mounted on the tip section where the diameter of the insertion section is desired to be reduced. Thus, a configuration in which the equalizer circuit 35 provided in the main body section 3 improves the waveform of the serial signal of the SLVS-EC serial communication (an SLVS-EC serial signal) as in the endoscope device 1 is a more preferable configuration.

Also, the equalizer circuit 35 corrects (improves) the waveform of the SLVS-EC serial signal so that similar signal levels are formed in all frequency bands by attenuating a signal component of a low-frequency component with a small amount of attenuation more to output the attenuated signal component when transmission is performed via the SLVS-EC serial signal transmission path 53. Thus, the SLVS-EC serial signal after the frequency characteristic is corrected by the equalizer circuit 35 becomes an overall low signal level (for example, several mV). Therefore, in the endoscope device 1, a configuration in which the corrected SLVS-EC serial signal output from the equalizer circuit 35 is amplified by the limiting amplifier circuit 36 is adopted.

The limiting amplifier circuit 36 is an amplification (amplifier) circuit configured to amplify the SLVS-EC serial signal after the frequency characteristic is corrected by the equalizer circuit 35. The limiting amplifier circuit 36 amplifies the signal level of the corrected SLVS-EC serial signal output from the equalizer circuit 35 to a level required for the multimedia processor 33 to perform image processing. Then, the limiting amplifier circuit 36 outputs the SLVS-EC serial signal whose signal level is amplified to the multimedia processor 33. For example, the limiting amplifier circuit 36 amplifies the signal level of the corrected SLVS-EC serial signal by a factor of 100 to several hundreds, and outputs the SLVS-EC serial signal with the amplified signal to the multimedia processor 33.

Also, as described above, because the SLVS-EC serial signal after the frequency characteristic is corrected by the equalizer circuit 35 generally has a low signal level, it is desirable to arrange the equalizer circuit 35 and the limiting amplifier circuit 36 so that they are close to each other in the endoscope device 1. That is, in the endoscope device 1, it is desirable to shorten the length of the signal line of the SLVS-EC serial signal between the equalizer circuit 35 and the limiting amplifier circuit 36 as much as possible.

Thus, in the endoscope device 1, the equalizer circuit 35 and the limiting amplifier circuit 36 are mounted on the same substrate surface of the main body substrate. In other words, in the endoscope device 1, soldering surfaces on which the equalizer circuit 35 and the limiting amplifier circuit 36 are soldered are on the same surface of the main body substrate, and signal lines of the equalizer circuit 35 and the limiting amplifier circuit 36 are connected on the same substrate surface of the main body substrate. Thereby, in the endoscope device 1, it is possible to ensure the quality of the waveform of the SLVS-EC serial signal without changing the characteristic impedance of the signal line between the equalizer circuit 35 and the limiting amplifier circuit 36.

Also, in the endoscope device 1, a substrate surface on which the equalizer circuit 35 is mounted (soldered), a substrate surface on which the cable constituting the SLVS-EC serial signal transmission path 53 is soldered, and a substrate surface on which the limiting amplifier circuit 36 is mounted (soldered) are preferably the same substrate surface of the main body substrate. However, as long as at least one of a case in which the substrate surface on which the equalizer circuit 35 is mounted and the substrate surface on which the cable constituting the SLVS-EC serial signal transmission path 53 is soldered become the same substrate surface and a case in which the substrate surfaces on which the equalizer circuit 35 and the limiting amplifier circuit 36 are mounted become the same substrate surface can be implemented in the endoscope device 1, the quality of the waveform of the SLVS-EC serial signal can be ensured.

Next, the components of the signal line and the transmission path provided in the flexible section 5 will be described in detail.

The power signal line 51 includes a single electric wire (a power cable). In the configuration of the single power cable, the power signal line 51 supplies power output from the power output section 32 provided in the main body section 3 to the power input section 411 provided in the image sensor 41 within the tip section 4.

The I2C serial signal transmission path 52 includes a set of twisted pair cables obtained by twisting two single lines corresponding to I2C serial signals. The I2C serial signal transmission path 52 implements the I2C serial communication between the multimedia processor 33 provided in the main body section 3 and the I2C communication section 413 provided in the image sensor 41 within the tip section 4 in the configuration of the single-line twisted pair cable.

Also, in the I2C serial signal transmission path 52, each of the two single lines is conceived to be formed as a shield line (a coaxial line) to prevent external noise from entering a single line corresponding to each I2C serial signal, i.e., to improve noise immunity. However, because the noise immunity of the I2C serial communication is improved by the stuck recovery circuit 34 provided in the main body section 3 in the endoscope device 1, a single line capable of narrowing the diameter of the I2C serial signal transmission path 52 can be used as a signal line corresponding to each I2C serial signal.

The SLVS-EC serial signal transmission path 53 includes a set of twisted pair cables obtained by twisting two shield lines (coaxial lines) corresponding to the SLVS-EC serial signals. The SLVS-EC serial signal transmission path 53 implements the SLVS-EC serial communication from the SLVS-EC output section 414 provided in the image sensor 41 within the tip section 4 to the multimedia processor 33 provided in the main body section 3 in the configuration of the single-line twisted pair cable. The SLVS-EC serial signal transmission path 53 includes the shield-line twisted pair cable because pixel signals are transmitted at a high bit rate of, for example, 1 to 2 gigabits/second (Gbps) or more in the SLVS-EC serial communication.

The diameter of the flexible section 5 can be narrowed according to configurations of the signal line and the transmission path. Thereby, the flexible section 5 can improve characteristics of insertion of the tip section 4 into the specimen. Thereby, in the endoscope device 1, a larger number of specimens can be designated as an object to be inspected, i.e., the width of the specimen can be increased.

According to the first embodiment, an endoscope device (the endoscope device 1) includes a tip section (the tip section 4) including an imaging element (the image sensor 41) for outputting a pixel signal according to a captured image of a subject and configured to be inserted into a specimen; a flexible section (the flexible section 5) including a first serial signal transmission path (the I2C serial signal transmission path 52) along which a setting related to photographing (a photographing mode setting) is transmitted to the imaging element (the image sensor 41) according to first serial communication (I2C serial communication) and which includes a stuck bus recovery circuit (the stuck recovery circuit 34) for performing a process of recovering the I2C serial communication that is stopped when the I2C serial communication is stopped and a second serial signal transmission path (the SLVS-EC serial signal transmission path 53) along which the pixel signal output by the imaging element (the image sensor 41) is transmitted according to second serial communication (SLVS-EC serial communication) and configured to guide the tip section 4 into the specimen, and a main body section (the main body section 3) including an image processing section (the multimedia processor 33) configured to perform image processing on the pixel signal transmitted according to the SLVS-EC serial communication.

Also, according to the first embodiment, the endoscope device 1 in which the SLVS-EC serial signal transmission path 53 includes an equalizer circuit (the equalizer circuit 35) configured to correct the frequency characteristic of the serial signal (the SLVS-EC serial signal) for transmitting the pixel signal according to the SLVS-EC serial communication and a limiting amplifier circuit (the limiting amplifier circuit 36) configured to amplify the SLVS-EC serial signal after the equalizer circuit 35 corrects the frequency characteristic is configured.

Also, according to the first embodiment, the endoscope device 1 in which the equalizer circuit 35 corrects a signal level of the input SLVS-EC serial signal for a signal output so that an attenuation rate of a signal level of a signal to be output (the SLVS-EC serial signal after correction) is decreased when a frequency band of an input signal (the SLVS-EC serial signal) is high and the attenuation rate of the signal level of the signal to be output (the SLVS-EC serial signal after correction) is increased when the frequency band of the input signal (the SLVS-EC serial signal) is low is configured.

Also, according to the first embodiment, the endoscope device 1 in which the I2C serial communication is serial communication through an I2C bus and the SLVS-EC serial communication is clock-embedded high-speed digital serial communication is configured.

Also, according to the first embodiment, the endoscope device 1 in which the SLVS-EC serial communication is the clock-embedded high-speed digital serial communication in which a synchronization signal (a horizontal synchronization signal or a vertical synchronization signal) indicating a timing at which the image sensor 41 outputs the pixel signal is embedded as a clock signal is configured.

Also, according to the first embodiment, the endoscope device 1 in which a cable for transmitting the pixel signal and the equalizer circuit 35 in the SLVS-EC serial signal transmission path 53 are connected on the same substrate surface (on the same substrate surface of the main body substrate) is configured.

Also, according to the first embodiment, the endoscope device 1 in which the equalizer circuit 35 and the limiting amplifier circuit 36 in the SLVS-EC serial signal transmission path 53 are connected on the same substrate surface (on the same substrate surface of the main body substrate) is configured.

Also, according to the first embodiment, the endoscope device 1 in which the I2C serial signal transmission path 52 is a transmission path having a narrower diameter than the SLVS-EC serial signal transmission path 53 is configured.

Also, according to the first embodiment, the endoscope device 1 in which the I2C serial signal transmission path 52 may include a single-line twisted pair cable corresponding to each signal in the I2C serial communication (a set of twisted pair cables obtained by twisting two single lines corresponding to the I2C serial signals), and the SLVS-EC serial signal transmission path 53 may include a shield-line twisted pair cable corresponding to each serial signal in the SLVS-EC serial communication (a set of twisted pair cables obtained by twisting two shield lines (coaxial lines) corresponding to the SLVS-EC serial signals) is configured.

As described above, in the endoscope device 1 of the first embodiment, the crystal oscillator 42 is provided in the tip section 4. Thereby, in the endoscope device 1 of the first embodiment, it is possible to narrow a diameter of the flexible section 5 by adopting a configuration in which a signal line for enabling the main body section 3 to supply an operation clock signal to the image sensor 41 provided in the tip section 4 is not provided in the flexible section 5.

Also, in the endoscope device 1 of the first embodiment, various settings (imaging mode settings) related to function activation and photographing operation of the image sensor 41 provided in the tip section 4 are performed through the I2C serial communication between the I2C communication section 413 provided in the image sensor 41 and the multimedia processor 33 provided in the main body section 3. Also, in the endoscope device 1 of the first embodiment, the stuck recovery circuit 34 is provided on the main body section 3 side of the I2C serial signal transmission path 52 which is a transmission path in the I2C serial communication. In the endoscope device 1 of the first embodiment, the stuck recovery circuit 34 monitors a state of the I2C serial communication between the I2C communication section 413 provided in the image sensor 41 within the tip section 4 and the multimedia processor 33 provided in the main body section 3 and causes the stopped I2C serial communication to be recovered (resumed) when it is determined that the I2C serial communication is stopped. Thereby, in the endoscope device 1 of the first embodiment, it is possible to improve the noise immunity of the I2C serial communication and narrow the diameter of the insertion section 2 even when the length of the I2C serial signal transmission path 52, which is the transmission path in the I2C serial communication, is, for example, a length exceeding 10 meters.

Also, in the endoscope device 1 of the first embodiment, the SLVS-EC output section 414 provided in the image sensor 41 transmits pixel signals obtained by performing photographing according to information of photographing mode settings in the image sensor 41 provided in the tip section 4 to the main body section 3 according to SLVS-EC serial communication. Also, in the endoscope device 1 of the first embodiment, the equalizer circuit 35 and the limiting amplifier circuit 36 are provided in the main body section 3 at the main body section 3 side of the SLVS-EC serial signal transmission path 53, which is a transmission path in the SLVS-EC serial communication. In the endoscope device 1 of the first embodiment, the equalizer circuit 35 corrects distortion of frequency characteristics of each serial signal (SLVS-EC serial signal) in the SLVS-EC serial communication for transmitting pixel signals obtained through photographing performed by the image sensor 41 from the SLVS-EC output section 414 provided in the image sensor 41 within the tip section 4. Also, in the endoscope device 1 of the first embodiment, the limiting amplifier circuit 36 amplifies a signal level of each SLVS-EC serial signal which is generally lowered through the correction of the distortion in the frequency characteristics in the equalizer circuit 35, and outputs the amplified signal to the multimedia processor 33. Thereby, even when the length of the SLVS-EC serial signal transmission path 53, which is the transmission path in the SLVS-EC serial communication, is, for example, a length exceeding 10 meters in the endoscope device 1 of the first embodiment, it is possible to accurately receive each pixel signal transmitted according to SLVS-EC serial communication and perform various types of image processing on each pixel signal.

Thereby, in the endoscope device 1 of the first embodiment, the number of signal cables provided within the flexible section 5 constituting the insertion section 2 can be reduced. More specifically, in the endoscope device 1 of the first embodiment, it is possible to narrow the diameter of the flexible section 5 because it is only necessary to provide five signal cables of the power signal line 51 including a single-line power cable, the I2C serial signal transmission path 52 including a single-line twisted pair cable, and the SLVS-EC serial signal transmission path 53 including a single-line twisted pair cable in the flexible section 5. In other words, in the endoscope device 1 of the first embodiment, it is possible to narrow the diameter of the flexible section 5 because the I2C serial signal transmission path 52 can be configured with a single-line twisted pair cable having a narrower diameter than a shield-line twisted pair cable constituting the SLVS-EC serial signal transmission path 53. In the endoscope device 1 of the first embodiment, the stuck recovery circuit 34 can improve the noise immunity of the I2C serial communication even when the diameter is narrowed by configuring the I2C serial signal transmission path 52 with a single-line twisted pair cable. Thereby, in the endoscope device 1 of the first embodiment, it is possible to improve noise immunity in a state in which the diameter of the insertion section 2 is narrowed. Thereby, in the endoscope device 1 of the first embodiment, it is possible to satisfy a requirement of electro-magnetic compatibility (EMC) even when the length of the insertion section 2 is, for example, a length exceeding 10 meters, in a state in which the diameter of the insertion section 2 is narrowed. When the length of the insertion section 2 is longer, it is more easily affected by external noise. Thus, when the endoscope device 1 is used in a place where an electromagnetic environment is significantly poor such as a factory, a shield line of the I2C serial signal transmission path 52 or the like may be necessary even if the stuck recovery circuit 34 exists. However, in such a case, as compared with when the stuck recovery circuit 34 is not provided, it is also possible to significantly reduce an increase in the outer diameter of the insertion section 2 by shielding the signal line.

Also, in the endoscope device 1 of the first embodiment, a configuration in which the insertion section 2 is integrated with the main body section 3, i.e., a configuration of the endoscope device 1 in which the insertion section 2 cannot be replaced and a distance to a subject within a specimen to be imaged is predetermined according to the length of the flexible section 5, is shown. However, the endoscope device 1 may be configured so that the insertion section 2 can be replaced.

### Second Embodiment

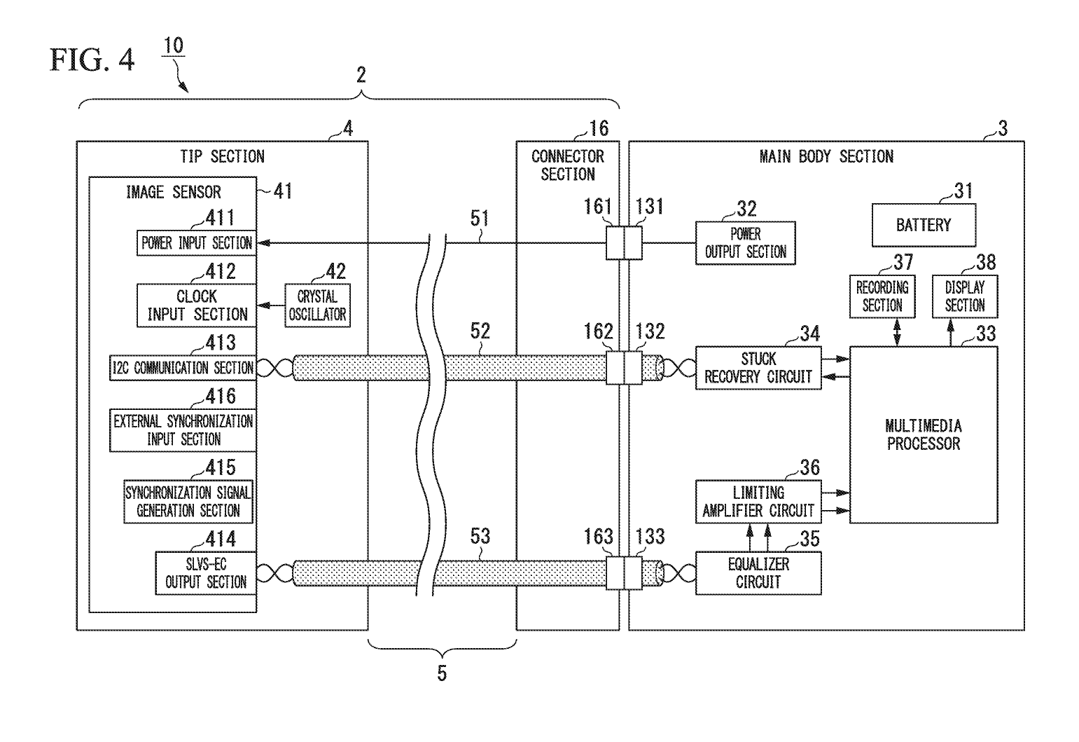

Next, an endoscope device of a second embodiment of the present invention will be described. Also, a case in which the endoscope device of the second embodiment is also an industrial endoscope device will be described. FIG. 4 is a block diagram showing an example of a schematic configuration of the endoscope device according to the second embodiment of the present invention. In FIG. 4, the endoscope device 10 includes an elongated insertion section 2 and a main body section 3. The insertion section 2 is configured to include a tip section 4 having an imaging element, a flexible section 5, which is a cord for guiding the tip section 4 into a specimen, and a connector section 16 for connecting the insertion section 2 to the main body section 3.

The endoscope device 10 shown in FIG. 4 is an endoscope device in which the insertion section 2 can be replaced in the endoscope device 1 of the first embodiment shown in FIG. 1. Accordingly, the endoscope device 10 according to the second embodiment includes components similar to those of the endoscope device 1 of the first embodiment shown in FIG. 1. In the following description, the same reference signs are given to components of the endoscope device 10 according to the second embodiment similar to those of the endoscope device 1 of the first embodiment, and a detailed description thereof will be omitted. In the following description, only components different from those of the endoscope device 1 of the first embodiment will be described.

In the endoscope device 10, the connector section 16 is provided on the main body section 3 side of the insertion section 2, and the insertion section 2 is configured so that the insertion section can be attached to or detached from the main body section 3 by the connector section 16. Then, in the endoscope device 10, a pixel signal obtained through photographing performed by the image sensor 41 provided within the tip section 4 is transmitted to the main body section 3 via the flexible section 5 and the connector section 16.

The connector section 16 includes an electrical contact point connector 161, an electrical contact point connector 162, and an electrical contact point connector 163. Also, the main body section 3 has a configuration in which an electrical contact point connector 131, an electrical contact point connector 132, and an electrical contact point connector 133 are added to the main body section 3 constituting the endoscope device 1 of the first embodiment.

The electrical contact point connector 161 is a connector corresponding to a power signal line 51 provided in the flexible section 5 and connected to the electrical contact point connector 131 provided in the main body section 3. Also, the electrical contact point connector 131 is a connector within the main body section 3 corresponding to the power signal line 51. By connecting the electrical contact point connector 161 and the electrical contact point connector 131, the power signal line 51 is electrically connected to a power output section 32 provided in the main body section 3. Thereby, in the endoscope device 10, power output from the power output section 32 is supplied to a power input section 411 provided in the image sensor 41 within the tip section 4 via the electrical contact point connector 131, the electrical contact point connector 161, and the power signal line 51.

The electrical contact point connector 162 is a connector corresponding to an I2C serial signal transmission path 52 provided in the flexible section 5 and connected to the electrical contact point connector 132 provided in the main body section 3. Also, the electrical contact point connector 132 is a connector within the main body section 3 corresponding to the I2C serial signal transmission path 52. In the endoscope device 10, the electrical contact point connector 162 and the electrical contact point connector 132 are connected and the I2C serial signal transmission path 52 is electrically connected to a stuck recovery circuit 34 provided in the main body section 3. Thereby, in the endoscope device 10, I2C serial communication is established between a multimedia processor 33 provided in the main body section 3 and an I2C communication section 413 provided in the image sensor 41 within the tip section 4 via the electrical contact point connector 132, the electrical contact point connector 162, and the I2C serial signal transmission path 52. In other words, in the endoscope device 10, the electrical contact point connector 162 and the electrical contact point connector 132 are connected and therefore various settings (photographing mode settings) related to function activation and photographing operation of the image sensor 41 are performed by the multimedia processor 33.

The electrical contact point connector 163 is a connector corresponding to the SLVS-EC serial signal transmission path 53 provided in the flexible section 5 and connected to the electrical contact point connector 133 provided in the main body section 3. Also, the electrical contact point connector 133 is a connector within the main body section 3 corresponding to the SLVS-EC serial signal transmission path 53. In the endoscope device 10, the SLVS-EC serial signal transmission path 53 is electrically connected to the equalizer circuit 35 provided in the main body section 3 by connecting the electrical contact point connector 163 and the electrical contact point connector 133. Thereby, in the endoscope device 10, the SLVS-EC serial communication from the SLVS-EC output section 414 provided in the image sensor 41 within the tip section 4 to the multimedia processor 33 provided in the main body section 3 is performed via the SLVS-EC serial signal transmission path 53, the electrical contact point connector 163, and the electrical contact point connector 133. In other words, in the endoscope device 10, pixel signals obtained through photographing performed by the image sensor 41 according to the information of the photographing mode settings are transmitted to the multimedia processor 33 by connecting the electrical contact point connector 163 and the electrical contact point connector 133.

Also, in the endoscope device 10, the equalizer circuit 35 and the electrical contact point connector 133 are mounted on the same substrate surface of the main body substrate. That is, in the endoscope device 10, soldering surfaces on which the equalizer circuit 35 and the electrical contact point connector 133 are soldered are on the same surface of the main body substrate, and signal lines of the equalizer circuit 35 and the electrical contact point connector 133 are connected on the same substrate surface of the main body substrate. Thereby, in the endoscope device 10, it is possible to ensure the quality of the waveform of the SLVS-EC serial signal without changing the characteristic impedance of the signal line between the equalizer circuit 35 and the electrical contact point connector 133. That is, in the endoscope device 10, it is possible to ensure the quality of the waveform of the SLVS-EC serial signal without changing the characteristic impedance of the signal line between the equalizer circuit 35 and the SLVS-EC serial signal transmission path 53 via the electrical contact point connector 133 and the electrical contact point connector 163.

In the endoscope device 10, as in the endoscope device 1 of the first embodiment, the equalizer circuit 35 and the limiting amplifier circuit 36 are also mounted on the same substrate surface of the main body substrate. In the endoscope device 10, it is desirable that a substrate surface on which the equalizer circuit 35 is mounted (soldered), a substrate surface on which the electrical contact point connector 133 is mounted (soldered), and a substrate surface on which the limiting amplifier circuit 36 is mounted (soldered) be the same substrate surface of the main body substrate. However, in the endoscope device 10, as in the endoscope device 1 of the first embodiment, it is possible to ensure the quality of the waveform of the SLVS-EC serial signal as long as at least one of a case in which the substrate surfaces on which the equalizer circuit 35 and the electrical contact point connector 133 are mounted become the same substrate surface and a case in which the substrate surfaces on which the equalizer circuit 35 and the limiting amplifier circuit 36 are mounted become the same substrate surface can be implemented.

According to such a configuration, in the endoscope device 10, a configuration in which the insertion section 2 can be replaced is implemented. Moreover, because the endoscope device 10 only includes electrical contact point connectors corresponding to the signal cables, it is possible to reduce the size of the connector section 16 and cost-effectively implement a configuration in which the insertion section 2 is replaced. Also, in the insertion section 2 having the short-length flexible section 5, reflection of a signal and distortion of a waveform of a signal generated when each signal passes through a corresponding electrical contact point connector are considered to be small. Thus, in the insertion section 2 having the short-length flexible section 5, it is possible to simplify the structure of the electrical contact point connectors provided in the connector section 16 and to further reduce the cost.

Also, the distortion of the waveform of the SLVS-EC serial signal for enabling the image sensor 41 to transmit the pixel signals according to the SLVS-EC serial communication is considered to vary with the length of the flexible section 5 constituting the insertion section 2. More specifically, the distortion of the frequency characteristics of the SLVS-EC serial signal in the SLVS-EC serial communication is considered to vary with the sum of lengths of the SLVS-EC serial signal transmission path 53, the electrical contact point connector 163, and the electrical contact point connector 133, i.e., the distance between the SLVS-EC output section 414 and the equalizer circuit 35. For example, the distortion of the waveform of the SLVS-EC serial signal is small if the length of the flexible section 5 is short and the distortion of the waveform of the SLVS-EC serial signal increases if the length of the flexible section 5 is long. Thus, in the amount of correction when the equalizer circuit 35 corrects frequency characteristics of the SLVS-EC serial signal, i.e., a frequency characteristic of the equalizer circuit 35, an optimum frequency characteristic varies with the length of the flexible section 5. Therefore, in the endoscope device 10, a configuration in which the frequency characteristic of the equalizer circuit 35 provided in the main body section 3 can change in accordance with the length of the flexible section 5 in the connected insertion section 2 is adopted. More specifically, the equalizer circuit 35 provided in the main body section 3 of the endoscope device 10 has a configuration in which constants of circuit elements provided in the equalizer circuit 35 can change according to settings from the multimedia processor 33. For example, in the configuration of the equalizer circuit 35 shown in FIG. 2, a configuration in which constants of circuit elements of a capacitor 351, two first resistors 352-1 and 352-2, a second resistor 353, and a coil 354 can change according to settings from the multimedia processor 33 is adopted. Thereby, in the equalizer circuit 35 provided in the main body section 3 of the endoscope device 10, for example, it is possible to change a frequency characteristic curve shown in FIG. 3 in accordance with the length of the flexible section 5 in the connected insertion section 2.

According to the second embodiment, the endoscope device (the endoscope device 10) further including a connector section (the connector section 16) configured to electrically connect the first serial signal transmission path (the I2C serial signal transmission path 52) and the second serial signal transmission path (the SLVS-EC serial signal transmission path 53) provided in the flexible section (the flexible section 5) to corresponding components (the stuck recovery circuit 34 and the equalizer circuit 35) provided in the main body section (the main body section 3) is configured.

As described above, in the endoscope device 10 of the second embodiment, as in the endoscope device 1 of the first embodiment, it is also possible to improve noise immunity in a state in which the diameter of the insertion section 2 is narrowed. That is, in the endoscope device 10 of the second embodiment, as in the endoscope device 1 of the first embodiment, it is possible to satisfy a requirement of EMC even when the length of the insertion section 2 is, for example, a length exceeding 10 meters, in a state in which the diameter of the insertion section 2 is narrowed. Moreover, in the endoscope device 10 of the second embodiment, it is possible to replace the insertion section 2.

Also, in the endoscope device 10 of the second embodiment, for example, a configuration in which the multimedia processor 33 changes the constants of the circuit elements provided in the equalizer circuit 35 provided in the main body section 3 and therefore a frequency characteristic curve in the equalizer circuit 35 can change in accordance with the length of the flexible section 5 in the connected insertion section 2 is implemented. However, if the endoscope device 1 is configured so that the insertion section 2 can be replaced, a configuration in which the frequency characteristic curve in the equalizer circuit 35 does not change, i.e., the equalizer circuit 35 of an optimum frequency characteristic for each insertion section 2 to be replaced is provided, may be adopted.

### Third Embodiment

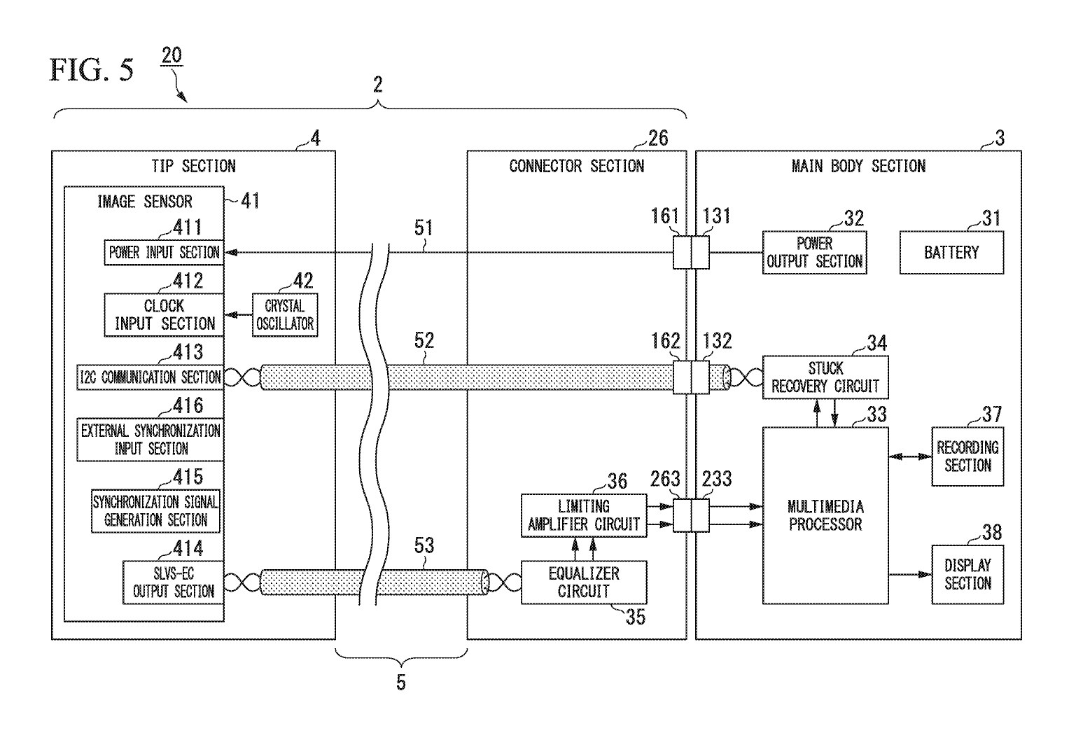

Next, an endoscope device of a third embodiment of the present invention will be described. A case in which the endoscope device of the third embodiment is also an industrial endoscope device will be described. FIG. 5 is a block diagram showing an example of a schematic configuration of an endoscope device according to a third embodiment of the present invention. In FIG. 5, the endoscope device 20 includes an elongated insertion section 2 and a main body section 3. The insertion section 2 is configured to include a tip section 4 having an imaging element, a flexible section 5 which is a cord for guiding the tip section 4 into a specimen, and a connector section 26 for connecting the insertion section 2 to the main body section 3.

The endoscope device 20 shown in FIG. 5 is an endoscope device having a configuration in which a frequency characteristic curve in the equalizer circuit 35 does not change in the endoscope device 10 of the second embodiment shown in FIG. 4. Accordingly, the endoscope device 20 according to the third embodiment includes components similar to those of the endoscope device 10 of the second embodiment shown in FIG. 4. In the following description, the same reference signs are given to the components of the endoscope device 20 according to the third embodiment similar to those of the endoscope device 10 of the second embodiment and a detailed description thereof will be omitted. In the following description, only components different from those of the endoscope device 10 of the second embodiment will be described.

In the endoscope device 20, a configuration in which the connector section 26 is provided on the main body section 3 side of the insertion section 2 and the insertion section 2 can be attached to or detached from the main body section 3 by the connector section 26 is adopted. Then, in the endoscope device 20, pixel signals obtained through photographing performed by the image sensor 41 provided within the tip section 4 are transmitted to the main body section 3 via the flexible section 5 and the connector section 26.

The connector section 26 includes an equalizer circuit 35, a limiting amplifier circuit 36, an electrical contact point connector 161, an electrical contact point connector 162, and an electrical contact point connector 263. Also, the main body section 3 includes an electrical contact point connector 131, an electrical contact point connector 132, and an electrical contact point connector 233.

The equalizer circuit 35 and the limiting amplifier circuit 36 are configured by arranging (moving) the equalizer circuit 35 and the limiting amplifier circuit 36 provided in the main body section 3 in the endoscope device 1 of the first embodiment and the endoscope device 10 of the second embodiment within the connector section 26. Accordingly, in the endoscope device 20, the electrical contact point connector 163 provided in the connector section 16 in the endoscope device 10 of the second embodiment is configured to be replaced with an electrical contact point connector 263. Also, in the endoscope device 20, a configuration in which the electrical contact point connector 133 provided in the main body section 3 in the endoscope device 10 of the second embodiment is replaced with an electrical contact point connector 233 is adopted.

In the connector section 26, each of two shield lines (coaxial lines) corresponding to SLVS-EC serial signals in the SLVS-EC serial signal transmission path 53 provided in the flexible section 5 is connected to the equalizer circuit 35 as in endoscope device 1 of the first embodiment. In the connector section 26, the frequency characteristic is corrected by the equalizer circuit 35 and each SLVS-EC serial signal whose signal level is amplified by the limiting amplifier circuit 36 is connected to the electrical contact point connector 263.

Also, in the endoscope device 20, when wire materials (cables) constituting the equalizer circuit 35 and the SLVS-EC serial signal transmission path 53 are connected in a substrate on which components constituting the connector section 26 are mounted (hereinafter referred to as a “connector substrate”), a substrate surface on which the equalizer circuit 35 is mounted (soldered) and a substrate surface on which cables constituting the SLVS-EC serial signal transmission path 53 are soldered become the same substrate surface. That is, in the endoscope device 20, the equalizer circuit 35 and the cables constituting the SLVS-EC serial signal transmission path 53 are connected on the same substrate surface of the connector substrate. Thereby, in the endoscope device 20, as in the endoscope device 1 of the first embodiment, it is also possible to ensure the quality of the waveform of the SLVS-EC serial signal without changing the characteristic impedance of the signal line between the equalizer circuit 35 and the SLVS-EC serial signal transmission path 53.

Also, in the endoscope device 20, as in the endoscope device 1 of the first embodiment and the endoscope device 10 of the second embodiment, the equalizer circuit 35 and the limiting amplifier circuit 36 are also mounted on the same substrate surface of the connector substrate. That is, in the endoscope device 20, soldering surfaces on which the equalizer circuit 35 and the limiting amplifier circuit 36 are soldered are on the same surface of the connector substrate and signal lines of the equalizer circuit 35 and the limiting amplifier circuit 36 are connected on the same substrate surface of the connector substrate. In the endoscope device 20, as in the endoscope device 1 of the first embodiment and the endoscope device 10 of the second embodiment, it is also desirable that a substrate surface on which the equalizer circuit 35 is mounted (soldered), a substrate surface on which the cable constituting the SLVS-EC serial signal transmission path 53 is soldered, and a substrate surface on which the limiting amplifier circuit 36 is mounted (soldered) be the same substrate surface of the connector substrate. However, in the endoscope device 20, as in the endoscope device 1 of the first embodiment, it is also possible to ensure the quality of the waveform of the SLVS-EC serial signal as long as at least one of a case in which a substrate surface on which the equalizer circuit 35 is mounted and a substrate surface on which the cable constituting the SLVS-EC serial signal transmission path 53 are soldered become the same substrate surface and a case in which the substrate surfaces on which the equalizer circuit 35 and the limiting amplifier circuit 36 are mounted become the same substrate surface can be implemented.

The electrical contact point connector 263 is a connector corresponding to each SLVS-EC serial signal whose signal level has been amplified output from the limiting amplifier circuit 36 and connected to the electrical contact point connector 233 provided in the main body section 3. Also, the electrical contact point connector 233 is a connector within the main body section 3 corresponding to each SLVS-EC serial signal whose signal level is amplified. When the electrical contact point connector 263 and the electrical contact point connector 233 are connected, each SLVS-EC serial signal whose signal level is amplified is electrically connected to the multimedia processor 33 provided in the main body section 3. That is, in the endoscope device 20, correction of distortion of the frequency characteristic and amplification on the SLVS-EC serial signal transmitted from the SLVS-EC output section 414 provided in the image sensor 41 within the tip section 4 via the SLVS-EC serial signal transmission path 53 are performed within the connector section 26 and the SLVS-EC serial signal after the correction and the amplification is input to the multimedia processor 33 provided in the main body section 3 via the electrical contact point connector 263 and the electrical contact point connector 233.

According to such a configuration, in the endoscope device 20, as in the endoscope device 10 of the second embodiment, a configuration in which the insertion section 2 can be replaced is implemented. Moreover, the endoscope device 20 performs correction of distortion of the frequency characteristic and amplification on the SLVS-EC serial signal for transmitting the pixel signals according to the SLVS-EC serial communication within the connector section 26 constituting the insertion section 2. Thus, although the endoscope device 10 of the second embodiment is affected by reflection of a signal and distortion of a waveform of a signal generated when each signal passes through a corresponding electrical contact point connector in the insertion section 2 having the long-length flexible section 5, it is possible to implement a configuration in which the insertion section 2 can be replaced in a state in which an influence on a signal when the signal passes through the electrical contact point connector is avoided in the endoscope device 20. In the endoscope device 20, the frequency characteristic of the equalizer circuit 35 configured to correct the distortion of the waveform of the SLVS-EC serial signal considered to vary with the length of the flexible section 5 constituting the insertion section 2 can be set to an optimum frequency characteristic for each insertion section 2. More specifically, the constant of each circuit element provided in the equalizer circuit 35 within the connector section 26 of the endoscope device 20 can be set to a constant for implementing an optimum frequency characteristic according to the length of the flexible section 5. For example, in the configuration of the equalizer circuit 35 shown in FIG. 2, the constant of each circuit element of the capacitor 351, the two first resistors 352-1 and 352-2, the second resistor 353, and the coil 354 can be set to a constant for implementing an optimum frequency characteristic according to the length of the flexible section 5. Thus, in the endoscope device 20, it is also possible to more accurately extract pixel signals of high-frequency components necessary when the multimedia processor 33 performs image processing.

According to the third embodiment, an endoscope device (the endoscope device 20) in which an equalizer circuit (the equalizer circuit 35) and a limiting amplifier circuit (the limiting amplifier circuit 36) are arranged in a connector section (the connector section 26) is configured.

As described above, in the endoscope device 20 of the third embodiment, as in the endoscope device 1 of the first embodiment and the endoscope device 10 of the second embodiment, it is possible to improve noise immunity in a state in which the diameter of the insertion section 2 is narrowed. That is, in the endoscope device 20 of the third embodiment, as in the endoscope device 1 of the first embodiment and the endoscope device 10 of the second embodiment, it is possible to satisfy a requirement of EMC even when the length of the insertion section 2 is, for example, a length exceeding 10 meters, in a state in which the diameter of the insertion section 2 is narrowed. In the endoscope device 20 of the third embodiment, as in the endoscope device 10 of the second embodiment, the insertion section 2 can be replaced. Moreover, although there is a possibility that the size of the connector section 26 constituting the insertion section 2 in the endoscope device 20 of the third embodiment will be slightly larger than the size of the connector section 16 constituting the insertion section 2 in the endoscope device 10 of the second embodiment, the equalizer circuit 35 within the connector section 26 can have an optimum frequency characteristic according to the length of the flexible section 5 and the transmitted pixel signal can be extracted more accurately.

As described above, according to each embodiment of the present invention, two types of serial signal transmission paths including a serial signal transmission path for performing various settings for the imaging element provided in the tip section located at a tip of the insertion section in the endoscope device according to serial communication and a serial transmission path for transmitting each pixel signal obtained through photographing performed by the imaging element provided in the tip section according to serial communication to the image processing section provided in the main body section in the endoscope device are provided. Thereby, in each embodiment of the present invention, it is possible to reduce the number of signal cables provided within the flexible section constituting the insertion section in the endoscope device. In each embodiment of the present invention, a stuck bus recovery circuit for performing a process of causing the stopped serial communication to be recovered (resumed) when the serial communication is stopped (stuck) is provided in the serial signal transmission path for performing settings for the imaging element. Thereby, in each embodiment of the present invention, it is possible to improve immunity against external noise entering the flexible section even when the diameter of the serial signal transmission path for performing settings for the imaging element provided within the flexible section constituting the insertion section in the endoscope device is narrowed. Also, in each embodiment of the present invention, the serial signal transmission path for transmitting each pixel signal includes the equalizer circuit configured to correct a frequency characteristic of a serial signal and the limiting amplifier circuit configured to amplify the serial signal after the equalizer circuit corrects the frequency characteristic are provided. Thereby, in each embodiment of the present invention, it is possible to accurately transmit each pixel signal to the image processing section even when the length of the flexible section constituting the insertion section in the endoscope device is long. Thereby, in each embodiment of the present invention, it is possible to implement the endoscope device having a long-length insertion section, which has noise immunity, i.e., satisfies a requirement of EMC, in a state in which the diameter of the insertion section is narrowed. Moreover, according to each embodiment of the present invention, it is possible to implement an endoscope device of a configuration in which the insertion section can be replaced in a state in which noise immunity is improved in a narrow diameter for the insertion section in the endoscope device.

Also, the case in which the endoscope device of the present invention is an industrial endoscope device has been described in each embodiment. However, the configuration and concept of each embodiment are not limited to application to an industrial endoscope device and, for example, may be similarly applied to a medical endoscope device. Thereby, in a medical endoscope device, it is also possible to obtain an effect similar to that of the industrial endoscope device described in each embodiment.

While preferred embodiments of the present invention have been described and shown above, it should be understood that these are exemplary of the invention and the present invention is not limited to these embodiments and modified examples thereof. Within a range not departing from the gist or spirit of the present invention, additions, omissions, substitutions, and other modifications to the configuration can be made.

Also, the present invention is not to be considered as being limited by the foregoing description, and is limited only by the scope of the appended claims.

What is claimed is:

1. An endoscope device, comprising:
a tip section including an imaging element for outputting a pixel signal according to a captured image of a subject and configured to be inserted into an object; and
a flexible section including a first serial signal transmission path along which a setting related to photographing is transmitted to the imaging element via first serial communication and a second serial signal transmission path along which the pixel signal output by the imaging element is transmitted via second serial communication and configured to guide the tip section into the object,
wherein the second serial signal transmission path includes an equalizer circuit configured to correct frequency characteristics of a serial signal for transmitting the pixel signal via the second serial communication and a limiting amplifier circuit configured to amplify the serial signal corrected by the equalizer circuit, and
wherein the equalizer circuit and the limiting amplifier circuit are connected on the same substrate surface.

2. The endoscope device according to claim 1, comprising:
a main body section including an image processing section configured to perform image processing on the pixel signal transmitted according to the second serial communication.

3. The endoscope device according to claim 2, further comprising:
a connector section configured to electrically connect the first serial signal transmission path and the second serial signal transmission path provided in the flexible section to corresponding components provided in the main body section.

4. The endoscope device according to claim 3, wherein the equalizer circuit and the limiting amplifier circuit are arranged in the connector section.

5. The endoscope device according to claim 1, wherein the equalizer circuit corrects a signal level of the input serial signal so that an attenuation rate of a signal level of a corrected signal becomes smaller when a frequency band of the input signal is higher, and that the attenuation rate of the signal level of the corrected signal becomes greater when the frequency band of the input signal is lower.

6. The endoscope device according to claim 1, wherein a cable for transmitting the pixel signal and the equalizer circuit in the second serial signal transmission path are connected on the same substrate surface.

7. The endoscope device according to claim 3, wherein a cable for transmitting the pixel signal and the equalizer circuit are arranged in the connector section.

8. An endoscope device, comprising:
a tip section including an imaging element for outputting a pixel signal according to a captured image of a subject and configured to be inserted into an object; and
a flexible section including a first serial signal transmission path along which a setting related to photographing is transmitted to the imaging element via first serial communication and a second serial signal transmission path along which the pixel signal output by the imaging element is transmitted via second serial communication and configured to guide the tip section into the object,
wherein the second serial signal transmission path includes an equalizer circuit configured to correct frequency characteristics of a serial signal for transmitting the pixel signal via the second serial communication, and
wherein a cable for transmitting the pixel signal and the equalizer circuit are connected on the same substrate surface.

9. The endoscope device according to claim 8, comprising:
a main body section including an image processing section configured to perform image processing on the pixel signal transmitted according to the second serial communication.

10. The endoscope device according to claim 8, wherein the second serial signal transmission path includes a limiting amplifier circuit configured to amplify the serial signal corrected by the equalizer circuit.

11. The endoscope device according to claim 10, wherein the equalizer circuit corrects a signal level of the input serial signal so that an attenuation rate of a signal level of a corrected signal becomes smaller when a frequency band of the input signal is higher, and that the attenuation rate of the signal level of the corrected signal becomes greater when the frequency band of the input signal is lower.

12. The endoscope device according to claim 10, wherein the equalizer circuit and the limiting amplifier circuit in the second serial signal transmission path are connected on the same substrate surface.

13. The endoscope device according to claim 9, further comprising:
a connector section configured to electrically connect the first serial signal transmission path and the second serial signal transmission path provided in the flexible section to corresponding components provided in the main body section.

14. The endoscope device according to claim 13, wherein the equalizer circuit and a limiting amplifier circuit configured to amplify the serial signal corrected by the equalizer circuit are arranged in the connector section.

15. The endoscope device according to claim 13, wherein the cable and the equalizer circuit are arranged in the connector section.
